(19) United States
(12) Patent Application Publication
(10) Pub. No.: US 2007/0004967 A1
(43) Pub. Date: Jan. 4, 2007

(54) Endoscope

(75) Inventors: Haruhiko Ueno, Tokyo (JP); Yutaka Masaki, Tokyo (JP); Shoichi Saito, Tokyo (JP)

Correspondence Address: Thomas Spinelli;Scully, Scott, Murphy & Presser, 400 Garden City Plaza, Garden City, NY, 11530, US

(73) Assignee: Olympus Corporation, Tokyo (JP)

(21) Appl. No.: 11/516,198

(22) Filed: Sep. 6, 2006

Related U.S. Application Data
(63) Continuation of application No. PCT/JP05/03524, filed on Mar. 2, 2005.

(30) Foreign Application Priority Data
Mar. 10, 2004 (JP) 2004-067903

(51) Int. Cl. A61B 1/00

(52) U.S. Cl. 600/152; 600/146

(57) ABSTRACT

An endoscope includes an insertion portion that has a bendable portion; and a bending manipulating device that bends the insertion portion by rotating a traction member extending from the bendable portion. The bending manipulating device includes a rotating member rotating with the traction member engaged, a driving motor that rotates the rotating member, and a speed reduction mechanism that transmits the driving force of the driving motor to the rotating member. The speed reduction mechanism includes a sun gear rotated by the driving force of the motor and having first external teeth, plural planetary gears each having second external teeth engaging with the first external teeth, a first gear having first internal teeth engaging with the second external teeth, and a second movable gear having second internal teeth engaging with the second external teeth. The number of second internal teeth is different from the number of first internal teeth.

### CROSS-REFERENCE TO RELATED APPLICATIONS

This application is a continuation of PCT international application Ser. No. PCT/JP2005/003524 filed Mar. 2, 2005 which designates the United States, incorporated herein by reference, and which claims the benefit of priority from Japanese Patent Application No. 2004-067903, filed Mar. 10, 2004, incorporated herein by reference.

### BACKGROUND OF THE INVENTION

1. Field of the Invention

The present invention relates to an endoscope that bends a bendable portion provided at an insertion portion by pulling and loosening a traction member extending from the bendable portion by driving a driving motor of a bending manipulating device.

2. Description of the Related Art

Conventionally, an endoscope is widely used in a medical field and an industrial field. In a general endoscope, a bendable portion that can be bent, for example, in upward and downward directions and in leftward and rightward directions, is provided at an elongated insertion portion. The bendable portion is made to bend when a traction member such as an angle wire that runs through inside the insertion portion is pulled and/or loosened through a manipulation of a manipulating lever provided in a manipulating portion.

Recently, an endoscope that is provided with an electric bending manipulating device in, for example, a manipulating portion thereof has been used to save power for a bending manipulation of the bendable portion. In the endoscope that is provided with the electric bending manipulating device, when a controller such as a joystick is tilted, a driving force of an electric motor causes pulling and loosening of a predetermined traction member. As a result, the bendable portion is bent.

For example, a bending manipulating device of an endoscope described in Japanese Patent No. 2660053 transmits rotational driving force of a motor disposed inside a manipulating portion to a gear on a driving side through a train of bevel gears. Then, torque of the gear on the driving side is transmitted to a gear on a driven side while a speed of the gear on the driving side is reduced. Since a sprocket is integrated with the gear on the driven side, the sprocket is rotated when the gear on the driven side is rotated. Consequently, a chain geared with the sprocket is moved, and the bendable portion is bent since one end of a wire that is secured to the chain is pulled and loosened.

### SUMMARY OF THE INVENTION

An endoscope according to one aspect of the present invention includes an insertion portion that has a bendable portion including plural bending pieces connected with each other; and a bending manipulating device that bends the insertion portion by rotating a traction member extending from the bendable portion and includes a rotating member rotating with the traction member engaged, a driving motor that rotates the rotating member, and a speed reduction mechanism that transmits the driving force of the driving motor to the rotating member. The speed reduction mechanism includes a sun gear rotated by the driving force of the motor and having first external teeth, plural planetary gears each having second external teeth engaging with the first external teeth, a first gear having first internal teeth engaging with the second external teeth, and a second movable gear having second internal teeth engaging with the second external teeth. The number of second internal teeth is different from the number of first internal teeth.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a drawing illustrating a configuration of an endoscope device;

FIG. 2 is a top view illustrating a configuration of a bending device;

FIG. 3 is a bottom view illustrating the configuration of the bending device;

FIG. 4 is a view illustrating gears configuring a major portion of a bending manipulating device;

FIG. 5 is a view illustrating a positional relationship of an arrangement and a gearing relationship of the gears of the bending manipulating device;

FIG. 6A is a view illustrating a detail of the switching mechanism and a relationship between a clutch lever and a fixed gear; and

FIG. 6B is a view illustrating the clutch lever.

### DETAILED DESCRIPTION OF THE PREFERRED EMBODIMENTS

Exemplary embodiments of the present invention will be explained below with reference to the accompanying drawings. An endoscope in which a bending manipulating device is installed in a manipulating portion is explained as an example in the explanation hereinafter. However, the present invention is not limited to the configuration described above, and, for example, the bending manipulating device and the manipulating portion can be provided separately.

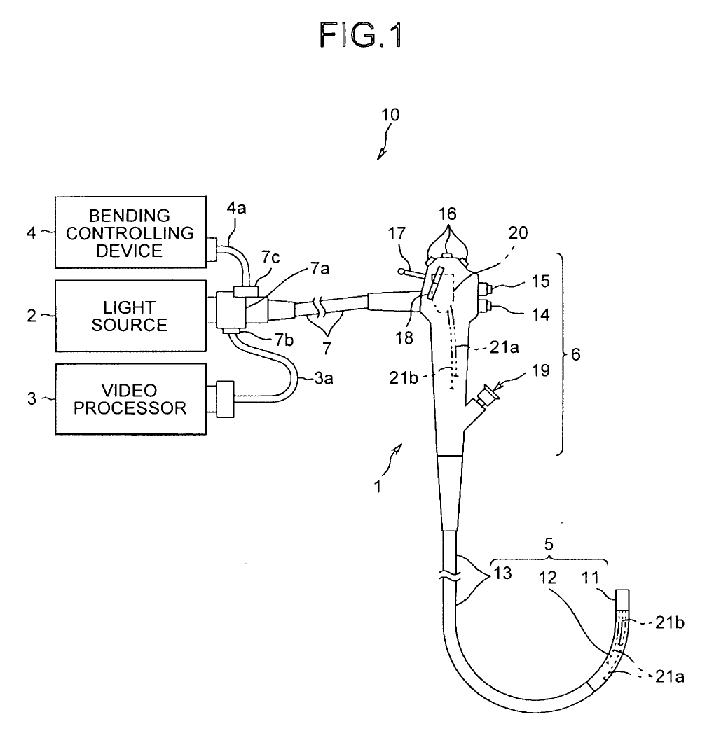

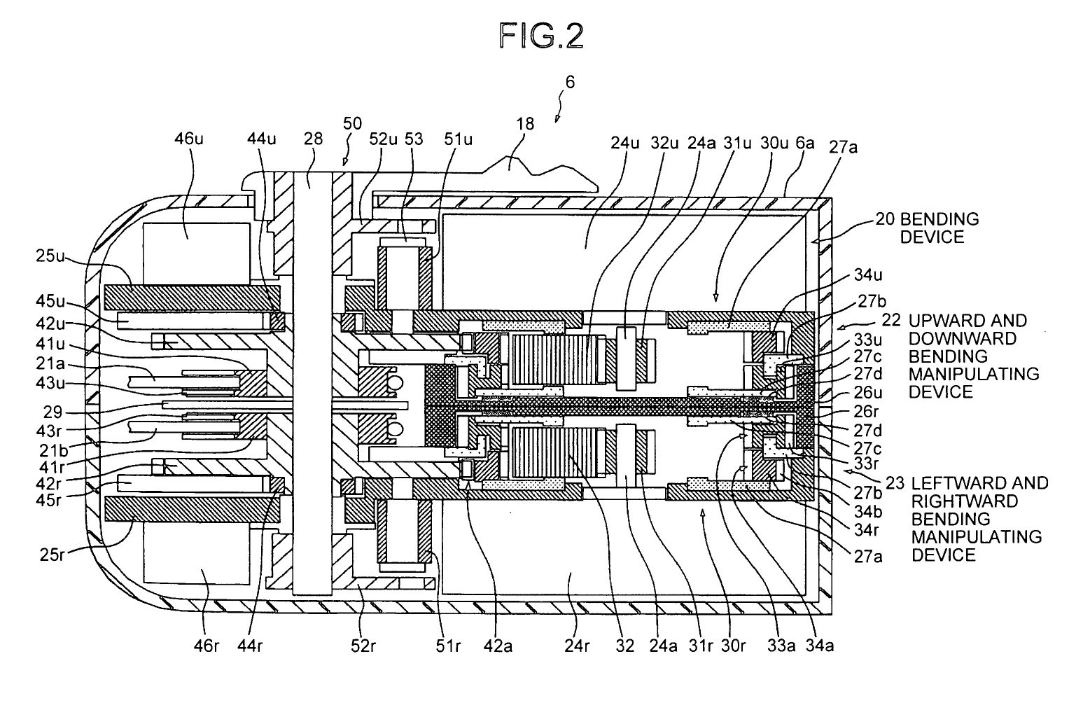

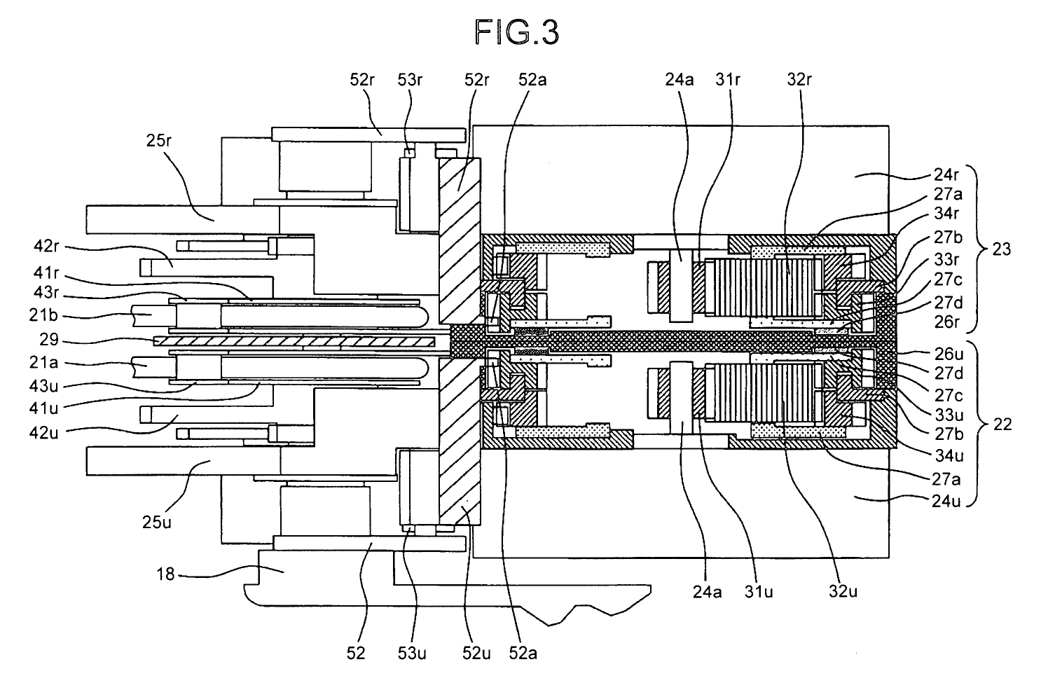

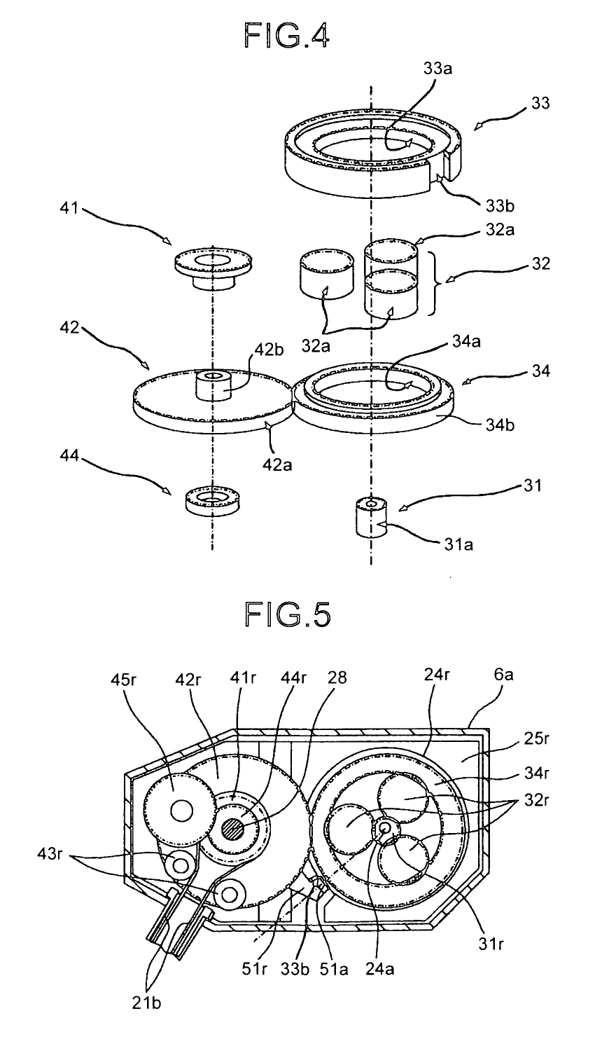

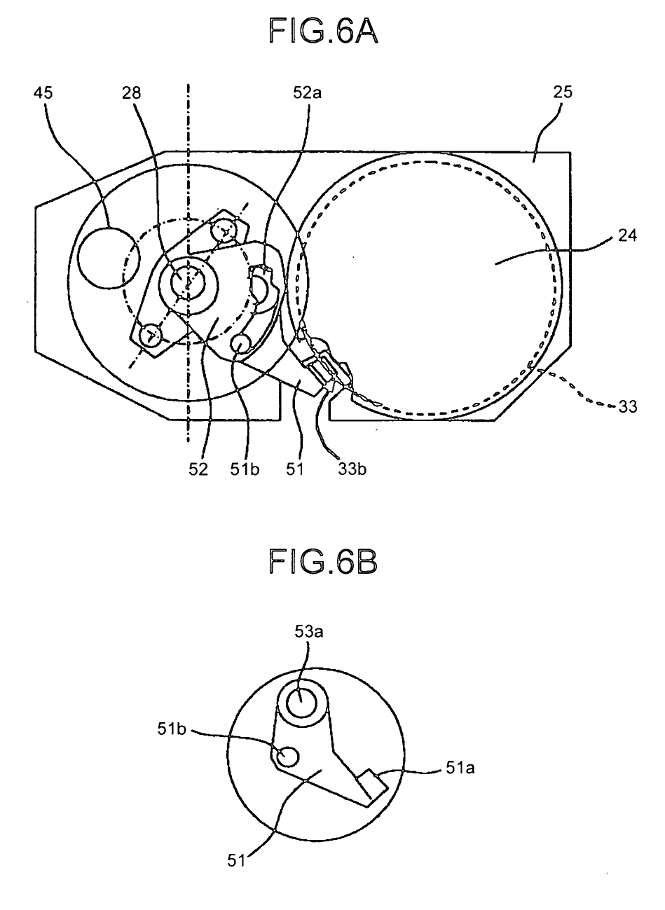

FIGS. 1 to 6 correspond to an embodiment of the present invention. FIG. 1 is a view illustrating a configuration of an endoscope device; FIG. 2 is a top view illustrating a configuration of a bending device; FIG. 3 is a bottom view illustrating the configuration of the bending device; FIG. 4 is a drawing illustrating gears configuring a major portion of a bending manipulating device; FIG. 5 is a drawing illustrating a positional relationship of an arrangement and a gearing relationship of the gears of the bending manipulating device; and FIGS. 6A and 6B are views illustrating a major portion of a switching mechanism.

FIG. 6A is a view illustrating a detail of the switching mechanism and a relationship between a clutch lever and a fixed gear, and FIG. 6B is a view illustrating the clutch lever.

As shown in FIG. 1, an endoscope device 10 having an endoscope 1 of the present invention mainly includes the endoscope 1; a light source 2 that supplies illumination light to the endoscope 1; a video processor 3 that performs a signal processing for an imaging device (not shown) installed in the endoscope 1; and a bending controlling device 4 that controls driving of a driving motor described later. The driving motor is a part of a later described bending device 20 provided in the endoscope 1.

The endoscope 1 includes an elongated insertion portion 5; a manipulating portion 6 that is provided at a proximal end side of the insertion portion 5 and also functions as a grip; and a universal cord 7 that extends from a side of the manipulating portion 6.

The insertion portion 5 has a rigid distal end portion 11; a bendable portion 12 that is bendable in directions of, for example, leftward and rightward and in directions of upward and downward, and connected to a proximal end side of the distal end portion 11; and a flexible pipe 13 having flexibility and connected to a proximal end side of the bendable portion 12. The distal end portion 11, the bendable portion 12, and the flexible pipe 13 are arranged in this order from the distal end side of the insertion portion 5.

The bendable portion 12 is configured to be bent in the upward and downward directions and in the leftward and rightward directions, by rotatably connecting plural bending pieces not shown to each other. An upward and downward direction manipulating wire 21a and leftward and rightward direction manipulating wire 21b are extended from the top bending piece of the bendable portion 12.

The manipulating portion 6 includes an air and water supply button 14 employed for water and air supply manipulation; an aspiration button 15 employed for aspiration manipulation; plural video switches 16 employed for remote control of the video processor 3; a device such as a joystick 17 outputting a command for pulling and loosening the wires 21a and 21b to bend the bendable portion 12; a manipulating lever 18 that is a part of a switching mechanism 50; and a surgical instrument insertion port 19 into which a surgical instrument such as a bioptome is inserted. The switching mechanism 50 switches between a state where the wires 21a and 21b are pulled due to the driving force of the driving motor provided in the bending device 20 and a state where the wires 21a and 21b are released from the pulling by the driving force of the driving motor. Here, proximal end portions of the wires 21a and 21b are disposed at the bending device 20.

A light source connector 7a that is detachably connected to the light source 2 is provided at an end of the universal cord 7. A video connector portion 7b and a bending controlling connector portion 7c are each provided on a side of the light source connector 7a. Here, a video cable 3a that is electrically connected to the video processor 3 is detachably connected to the video connector portion 7b, and an electric cable 4a that is electrically connected to the bending controlling device 4 is detachably connected to the bending controlling connector portion 7c.

A configuration and an effect of the bending device 20 are explained with reference to FIGS. 2 to 6.

As shown in FIGS. 2 and 3, the bending device 20 disposed inside an exterior case body 6a of the manipulating portion 6 includes a pair of bending manipulating devices, i.e., an upward and downward bending manipulating device 22 and a leftward and rightward bending manipulating device 23; and the switching mechanism 50.

Configurations and effects of the leftward and rightward bending manipulating device 23 and the upward and downward bending manipulating device 22 are substantially the same. Hence, when the configurations and the effects of the upward and downward bending manipulating device 22 and the leftward and rightward bending manipulating device 23 are explained, the configuration and the effect of the upward and downward bending manipulating device 22 are mainly explained, and the explanation is not to be repeated for the configuration and the effect of the leftward and rightward bending manipulating device 23.

Respective members constituting the upward and downward bending manipulating device 22 and the leftward and rightward bending manipulating device 23 are explained without letters for distinguishing members for upward and downward from members for leftward and rightward. When it is necessary to distinguish the members for the upward and downward bending manipulating device 22 from the members for the leftward and rightward bending manipulation device 23, a letter “u” is affixed to the reference character for each member for the upward and downward bending manipulation device 22, and a letter “r” is affixed for each member for the leftward and rightward bending manipulation device 23. Further, in the drawings, the letter “u” is affixed to the reference character of each member for the upward and downward bending manipulation device 22, and the letter “r” is affixed for each member for the leftward and rightward bending manipulation device 23 in order to distinguish the members for the upward and downward bending manipulation device 22 from the members for the leftward and rightward bending manipulation device 23.

The bending manipulating device 22 mainly includes a driving motor 24 (hereinafter referred to as motor), a speed reduction mechanism 30, and a rotating member 41.

The motor 24 has a flat shape and is secured to an exterior case body 6a side of a flame 25 having a step shape.

A configuration of the speed reduction mechanism 30 is explained in detail with reference to FIGS. 2 to 4 The speed reduction mechanism 30 mainly includes a sun gear 31 that consists of a spur gear disposed on a motor shaft 24a of the motor 24; three planetary gears 32, 32, and 32 that are, for example, spur gears; a fixed gear 33 that is a first gear and consists of an internal gear; and a movable gear 34 that is a second gear and consists of an internal gear. The sun gear 31, the planetary gears 32, 32, 32, the fixed gear 33, and the movable gear 34 are disposed in a case that consists of the flame 25 and a cap 26.

The movable gear 34 has a substantially ring shape and is disposed at a motor 24 side. The movable gear 34 has internal teeth 34a including a predetermined number of teeth formed on an inner circumferential face side thereof and external teeth 34b including a predetermined number of teeth formed on an outer circumferential face side thereof. The movable gear 34 is disposed between a first thrust receiver 27a and a second thrust receiver 27b so that the movable gear 34 can rotate around the motor shaft 24a as a central axis. The first thrust receiver 27a is securely fixed to the flame 25. The second thrust receiver 27b is disposed so as to be held between the cap 26 and the flame 25.

The fixed gear 33 has a ring shape which is substantially the same as that of the movable gear 34. The fixed gear 33 has internal teeth 33a having a predetermined number of teeth provided on an inner circumferential face side of the fixed gear 33, and an engaging groove 33b having a depressed shape formed on an outer circumferential face side thereof. The number of teeth in the internal teeth 33a of the fixed gear 33 differs from the number of teeth in the internal teeth 34a of the movable gear 34 by a predetermined amount. In the present embodiment, since there are three planetary gears, the difference between the numbers of teeth is three. For example, the number of teeth in the internal teeth 33a of the fixed gear 33 is set to be greater than the number of teeth in the internal teeth 34a of the movable gear 34.

The difference between the number of teeth in the internal teeth 33a of the fixed gear 33 and the number of teeth in the internal teeth 34a of the movable gear 34 is set to four (when there are four planetary gears) or six (when there are six planetary gears) corresponding to torque. Further, a shape of the internal teeth 33a of the fixed gear 33 is projected onto a shape of the internal teeth 34a of the movable gear 34 so that the planetary gear 32 gears with the internal teeth 33a of the fixed gear 33 and with the internal teeth 34a of the movable gear 34 while keeping an identical center distance.

The fixed gear 33 is also disposed between the second thrust receiver 27b and a third thrust receiver 27c so as to rotate around the motor shaft 24a as a central axis. An engaging portion 51a, which is provided on a rotatable clutch lever 51, engages with an engaging groove 33b of the fixed gear 33.

When the endoscope of the present embodiment is used in a normal state, the engaging portion 51a of the clutch lever 51 is engaged with the engaging groove 33b of the fixed gear 33 that is rotatably disposed between the second thrust receiver 27b and the third thrust receiver 27c. Consequently, the rotatably disposed gear functions as the fixed gear 33 as described above.

The third thrust receiver 27c is installed securely on the cap 26. The letter 27d represents an elastic member (spring member in the present embodiment) that is a member biasing the third thrust receiver 27c toward the motor 24. The bendable portion 12 that has been bent is prevented from rapidly coming back to straighten when the engaging portion 51a of the clutch lever 51 that has been engaged with the engaging groove 33b of the fixed gear 33 is disengaged, since the spring member 27d biases the third thrust receiver 27c.

Outside diameters of the fixed gear 33 and the movable gear 34 are substantially identical to or smaller than an outside diameter of the motor 24. Consequently, the device can be downsized by reducing a center distance between the motor shaft 24a and a rotating member supporting shaft 28 described hereinafter that is a supporting shaft of the rotating member 41.

External teeth 32a are formed on the planetary gear 32. The external teeth 32a engage with the external teeth of the sun gear 31, as well as gear with the internal teeth 33a of the fixed gear 33 and the internal teeth 34a of the movable gear 34. That is to say, a tooth width of the planetary gear 32 is such that the planetary gear 32 engages with each of the internal teeth 33a of the fixed gear 33 and the internal teeth 34a of the movable gear 34. The planetary gears 32, 32, and 32 are rotatably disposed between the first thrust receiver 27a and the third thrust receiver 27c.

An effect of the speed reduction mechanism 30 that is configured as described above is explained.

When the motor 24 is driven, rotational driving force of the motor 24 is transmitted to the movable gear 34 as described below.

First, the sun gear 31 that is disposed on the motor shaft 24a is rotated when the motor 24 is driven. Next, the rotation of the sun gear 31 is transmitted to the planetary gears 32, 32, and 32. Then, the planetary gears 32, 32, and 32 are started to rotate. At the moment, the engaging portion 51a is engaged with the engaging groove 33b in order not to rotate the fixed gear 33. Consequently, the planetary gears 32, 32, and 32 that are engaged with the fixed gear 33 and the movable gear 34 start to revolve around the sun gear 31 with the rotations thereof. That is to say, the planetary gears 32, 32, and 32 start to rotate and revolve by torque transmitted from the sun gear 31.

Here, the internal teeth 33a of the fixed gear 33 and the internal teeth 34a of the movable gear 34 are both geared with the external teeth 32a of the planetary gears 32, 32, and 32. The number of internal teeth 33a of the fixed gear 33 is different from the number of internal teeth 34a of the movable gear 34 by a predetermined number. Hence, a geared internal tooth 33a of the fixed gear 33 is shifted with respect to an internal tooth 34a of the movable gear 34 that is engaged with the external teeth 32a of the planetary gear 32. During the rotation and revolution of the planetary gears 32, 32, and 32, the movable gear 34 rotates in such a manner as to compensate for the shifting.

The movable gear 34 is continued to rotate with a reduced speed compared to the rotation of the motor 24 by continuously driving the motor 24 and by rotating and revolving the planetary gears 32, 32, and 32.

A configuration around the rotating member 41 is explained with reference to FIGS. 2 to 5.

The wire 21a or the wire 21b is integrally fixed to the rotating member 41. The rotating member 41 is integrally fixed to a large-diameter shaft 42b formed on a rotating external gear 42 that is a transmitting gear having external teeth 42a. The external teeth 42a engages with the external teeth 34b of the movable gear 34. The rotating external gear 42 that is integrated with the rotating member 41 is rotatably disposed with respect to the rotating member supporting shaft 28 in such a way that the rotating external gear 42 is arranged between the flame 25u and a separating board 29.

Consequently, the movable gear 34 and the rotating external gear 42 are prevented from being arranged so as to overlap each other with respect to the motor 24 since a position of the rotating member supporting shaft 28 and a position of the motor shaft 24a of the motor 24 are different. Therefore, a dimension in an extending direction of the motor shaft 24a of the motor 24 is suppressed from becoming large. That is to say, a width (thickness) of the manipulating portion is suppressed from becoming large.

When the motor 24 is continuously driven, the planetary gears 32, 32, and 32 are continued to rotate and revolve. Consequently, the movable gear 34 is rotated, and the rotating external gear 42 having the external teeth 42a that engage with the external teeth 34b of the movable gear 34 is rotated. Then, the rotating member 41 that is integrally secured to the rotating external gear 42 is rotated. Therefore, the wires 21a and 21b that are integrally disposed on the rotating member 41 are pulled and loosened. Each of the wires 21a and 21b that are integrally disposed on the rotating member 41 is held by a guide roller 43 disposed on the separating board 29.

Since the rotating members 41u and 41r that respectively form a part of the upward and downward bending manipulating device 22 and the leftward and rightward bending manipulating device 23 are arranged with the separating board 29 therebetween, the rotating members 41u and 41r do not come into contact with each other, and similarly, the wires 21a and 21b that are respectively attached to the rotating members 41u and 41r do not come into contact with each other.

A configuration of the switching mechanism 50 is explained with reference to FIGS. 2 to 6.

One end portion of the rotating member supporting shaft 28 is protruded from the exterior case body 6a, and the manipulating lever 18 is attached to the protruded portion.

The switching mechanism 50 is provided with the manipulating lever 18. The engaging portion 51a of the clutch lever 51 is engaged with the engaging groove 33b formed on the fixed gear 33 or disengaged from the engaging groove 33b by manipulating the manipulating lever 18.

The fixed gear 33 that is fixed as described above is rotatably held between the second thrust receiver 27b and the third thrust receiver 27c when the engaging portion 51a is disengaged from the engaging groove 33b. Consequently, the driving force of the motor 24 is not transmitted to the rotating member 41. That is to say, it is impossible to bend the bendable portion 12 by tilting the joystick 17. Such a state is called an angle-free state.

Specifically, the switching mechanism 50 includes the manipulating lever 18, a clutch cam 52, the clutch lever 51, and a clutch lever shaft 53.

The engaging portion 51a and a clutch pin 51b are provided on the clutch lever 51. The clutch lever 51 is integrally secured to a shaft 53a of the clutch lever shaft 53. A cam groove 52a is formed on the clutch cam 52. The clutch cam 52 is integrally secured to the rotating member supporting shaft 28. Further, the manipulating lever 18 is integrally secured to the clutch cam 52.

Hence, the clutch cam 52 is rotated in response to the manipulation of the manipulating lever 18. When the clutch cam 52 is rotated, the clutch pin 51b moves from one end portion side of the cam groove 52a to another end portion side of the cam groove 52b.

Consequently, a position of the clutch lever 51 secured on the clutch lever shaft 53 is changed, and the engaging portion 51a that is provided on the clutch lever 51 is shifted until the engaging portion 51a is disengaged from the engaging groove 33b. Then, the fixed gear 33 that is fixed between the second thrust receiver 27b and the third thrust receiver 27c starts to rotate.

Thus, the manipulation of the manipulating lever 18 allows switching between a state where the engaging portion 51a of the clutch lever 51 is fitted into the engaging groove 33b of the fixed gear 33 and a state where the engaging portion 51a is out of the engaging groove 33b.

In the present embodiment, the clutch lever 51 includes the engaging portion 51a and the fixed gear 33 includes the engaging groove 33b which corresponds to the engaging portion 51a, and form the switching mechanism. When the engaging portion 51a is engaged with the engaging groove 33b, the fixed gear 33 is brought into a fixed state. A structure that brings the fixed gear 33 into the fixed state is not limited to the combination of the engaging portion 51a and the engaging groove 33b. A member having a high friction coefficient, such as an elastic member, may be arranged on the outer circumferential face of the fixed gear 33, and a pressing button (not shown) provided on the clutch lever 51 may be pushed to press the high friction coefficient member to bring the fixed gear 33 into a fixed state. As far as the switching between the engaged state and the disengaged state can be performed through the manipulation of the manipulating lever 18, any structures can be employed.

A bending control of the bendable portion 12 is briefly explained.

Reference character “44” represents a first potentiometer gear. The first potentiometer gear 44 is integrally secured to the rotating external gear 42. Thus, when the rotating external gear 42 is rotated by the movable gear 34, the first potentiometer gear 44 is rotated accordingly.

A second potentiometer gear 45 is engaged with the first potentiometer gear 44. Therefore, the second potentiometer gear 45 is rotated by the rotation of the first potentiometer gear 44. The rotation of the second potentiometer gear 45 is detected by a potentiometer 46. A signal detected by the potentiometer 46 is a signal for calculating an advance and retreat amount of the wires 21a and 21b, and the signal is transmitted through a potentiometer signal cable not shown extending from the potentiometer 46.

The potentiometer signal cable is inserted into the universal cord 7 and extends to the light source connector 7a. Further, the bending controlling connector portion 7c of the light source connector 7a and the bending controlling device 4 are electrically connected by an electric cable 4a. Therefore, a rotated position detecting signal, which shows a rotated position output from the potentiometer 46, is input to a control unit not shown provided at the bending controlling device 4 through the potentiometer signal cable and the electric cable 4a.

Further, an encoder not shown, which is a rotated position detecting unit that serves to detect the rotated position of the motor shaft 24a of the motor 24, is provided at the manipulating portion 6. An encoder signal cable not shown extending from the encoder is inserted into the universal cord 7, and extends to the light source connector 7a. Then, the bending controlling connector portion 7c of the light source connector 7a and the bending controlling device 4 are electrically connected to each other by the electric cable 4a. Consequently, the rotated position detecting signal showing the rotated position of the motor shaft output from the encoder is input to the control unit not shown provided at the bending controlling device 4 through the encoder signal cable and the electric cable 4a.

Furthermore, a bending manipulating command signal showing a tilt angle and a tilt direction of the joystick 17 is output from the joystick 17 provided on the manipulating portion 6. The bending manipulating command signal is output to the control unit of the controlling device 4 by the manipulating portion signal cable extending from the joystick 17. Here, the manipulating portion signal cable is inserted into the universal cord 7, and extends to the light source connector 7a. Then, the bending controlling connector portion 7c of the light source connector 7a and the bending controlling device 4 are electrically connected to each other by the electric cable 4a.

On the other hand, a motor signal cable not shown extends from the motor 24. The motor signal cable is inserted into the universal cord 7, and the motor signal cable extends to the light source connector 7a. Further, the bending controlling connector portion 7c of the light source connector 7a and the bending controlling device 4 are electrically connected to each other by the electric cable 4a. Hence, a motor driving signal output from the control unit not shown provided on the bending controlling device 4 is output to the motor 24 through the electric cable 4a and the motor signal cable.

That is to say, the control unit outputs the motor driving signal to the motor 24 to drive control the motor 24 and thereby bending the bendable portion 12 based on the bending manipulating command signal output from the joystick 17 and based on the rotated position detecting signal output from the encoder and the potentiometer.

An effect of the bending device 20 provided in the manipulating portion 6 of the endoscope 1 configured as described above is explained.

An endoscopy and the like are performed by using the endoscope device 10 while the endoscope 1, the light source 2, the video processor 3, and the bending controlling device 4 are connected as explained with reference to FIG. 1. In such a connection, an operator inserts the distal end portion 11 of the insertion portion 5 towards a target site inside a body cavity while tilting the joystick 17 to bend the bendable portion 12 of the endoscope 1.

When the joystick 17 is tilted, the bending manipulating command signal is output towards the control unit from the joystick 17. Then, the control unit calculates an amount of traction, i.e., a required amount of movement of the bending wire, i.e., an amount of motor rotation, from the bending manipulation command signal. The control unit outputs a motor driving signal corresponding to the calculated value to the motor 24. Then, the motor shaft 24a of the motor 24 is brought into a rotated state.

Then, the sun gear 31 that is disposed at the motor shaft 24a is rotated, and the rotation of the sun gear 31 is transmitted to the planetary gears 32, 32, and 32. Consequently, the planetary gears 32, 32, and 32 start to rotate. At the moment, the fixed gear 33 is not rotated; therefore, the planetary gears 32, 32, and 32 that are engaged with the fixed gear 33 and the movable gear 34 start to rotate and revolve. Since the number of internal teeth 33a of the fixed gear 33 differs from the number of internal teeth 34a of the movable gear 34 by a predetermined number (here, the internal teeth 33a and the internal teeth 34a are the external teeth 32a of the planetary gears 32, 32, and 32), a geared internal tooth 33a becomes shifted with respect to a geared internal tooth 34a. Then, the movable gear 34 rotates at a reduced speed so as to compensate for the shifting.

Then, the rotating external gear 42 having the external teeth 42a that engage with the external teeth 34b of the movable gear 34 is rotated, and the rotating member 41 that is integrally secured to the rotating external gear 42 is rotated. Consequently, the wires 21a and/or 21b are pulled and loosened, and the bendable portion 12 starts to bend.

Here, the first potentiometer gear 44 and the second potentiometer gear 45 are rotated since the rotating external gear 42 is rotated. Since the rotation of the second potentiometer gear 45 is detected by the potentiometer 46, the signal detected by the potentiometer 46 is supplied to the control unit. When the control unit determines that the wires 21a and 21b are advanced or retreated by an amount corresponding to the tilt angle and the tilt direction of the joystick 17, the output of the motor driving signal that is output towards the motor 24 from the control unit is stopped as well as current supplied towards the motor 24 is stopped. Consequently, a desired bending state is obtained.

When an operator determines that the motor 24 might be out of control due to some influences while bending the bendable portion 12 of the endoscope 1, the operator rotates the manipulating lever 18 towards a predetermined direction in order to set the bendable portion 12 in the angle free state. Then, the engaging portion 51a of the clutch lever 51 is disengaged from the engaging groove 33b of the fixed gear 33 in conjunction with the movement of the manipulating lever 18, and the fixed gear 33 that had been fixed starts to rotate. Then, the fixed gear 33 is rotated, and the rotation of the movable gear 34 is stopped. Here, the planetary gears 32, 32, and 32 continue to rotate and revolve because the movable gear 34 obtains load (a) from the wire at the bendable portion 12, and because the fixed gear 33 obtains load (b) from the spring member 27d. Since an amount of the load is a>b, the fixed gear 33 is rotated and the rotation of the movable gear 34 is stopped. That is to say, the bending of the bendable portion 12 is stopped since the transmission of the rotation of the motor shaft 24a to the rotating member 41 is stopped.

When the fixed gear 33 is switched into a rotatable state, the bendable portion 12 is forced to change its state from the bent state into the straightened state due to the rotation of the fixed gear 33 and the movable gear 34 both engaged with the planetary gears 32. However, since the fixed gear 33 is biased by the spring member 27 and the thrust receiver 27c, the fixed gear 33 rotates only at a low speed. Therefore, the state of the bendable portion 12 does not change abruptly, but gradually changes from the bent state to an original, straightened state.

Since the speed reduction mechanism includes the sun gear; the planetary gears; the fixed gear that is the first gear gearing with the planetary gears; and the movable gear that is the second gear having a different number of teeth from the number of teeth of the fixed gear by the predetermined number, the rotational driving force of the sun gear that is rotated by the driving force of the motor can be transmitted by a combination of small gears at a large speed reduction ratio with respect to the movable gear. Consequently, a smaller speed reduction mechanism can be obtained compared with a speed reduction mechanism that includes a large number of gears to have a sufficient speed reduction ratio. Hence, the bending manipulating device in which the speed reduction mechanism is installed can be downsized as well as the manipulating portion can be downsized when the bending manipulating device is provided inside the manipulating portion. Specifically, in a speed reduction mechanism including plural planetary gears, outer diameters of the planetary gears can be decreased since physical strength required for each of the planetary gears is reduced compared to the physical strength required in a speed reduction mechanism including only a single gear. Therefore, the speed reduction mechanism can be further downsized.

Further, the endoscope can be manufactured with low cost because the gears constituting the speed reduction mechanism, i.e., the sun gear, the planetary gears, the fixed gear that is the first gear, and the movable gear that is the second gear, are spur gears that have good workability and do not require high accuracy in gearing.

Furthermore, since the external teeth provided in the movable gear are made to engage with the external teeth of the rotating external gear which is integrally formed with the rotating member, the rotating member supporting shaft is not arranged on an extended line of the motor shaft. Consequently, the bending manipulating device can be downsized, and the manipulating portion can be downsized when the bending manipulating device is provided in the manipulating portion.

Further, since the motor is made flat, the thickness of the manipulating portion that has an aligned structure of the upward and downward bending manipulating device and the leftward and rightward bending manipulating device can be decreased. Further, since the motor has a small outside diameter, the manipulating portion can be downsized by decreasing the distance between the rotating external gear and the rotating member. Consequently, freedom of layout is largely improved by shortening the length of the manipulating portion; adjusting the width of the manipulating portion and miniaturizing the manipulating portion; optimally changing an arrangement (balance) of the manipulating portion; or increasing an internal space inside the manipulating portion.

Furthermore, a spacing between the upward and downward wire and the leftward and rightward wire can be decreased by disposing the movable gear at a motor side with respect to the fixed gear and by arranging the upward and downward rotating member and the leftward and right ward rotating member close to each other while having the separating board therebetween. Consequently, a manipulating portion distal end side can be thinned down as well as the dimension of the bending manipulating device in the direction of the motor shaft can be miniaturized.

As is apparent from the figures, particularly FIG. 2, it is preferred to configure any of the upward and downward bending manipulating device 22 and the leftward and rightward bending manipulating device 23 in such a way that the rotating member can be arranged near an axis that is extended from the central axis of the insertion portion, in order to achieve the downsizing. The traction member (wires 21a and 21b) can be extended coaxially with an axis obtained by extending the central axis of the insertion portion extended from the insertion portion to the manipulating portion. Hence, increasing the spacing between the traction members relating to each of the upward and downward, and leftward and rightward bending manipulation devices can be avoided. This is an advantage that suppresses increasing the shape of the manipulating portion proximal end portion (end portion of insert portion side). Since both of the upward and downward bending manipulating device 22 and the leftward and rightward bending manipulating device 23 have such an advantage, the upward and downward bending manipulating device 22 and the leftward and rightward bending manipulating device 23 are preferably configured to form the rotating member to be arranged at the end portion in the direction of the motor shaft as well as to arrange the rotating member at a position near the axis that is extended from the central axis of the insertion portion.

On the other hand, an object of the present embodiment is to downsize the bending manipulating device. Hence, it is preferred to reduce the dimension thereof in the direction of the motor shaft as much as possible. Further, in the present embodiment, the transmitting gear (rotating external gear 42) is employed as the transmitting mechanism from the speed reduction mechanism to the rotating member, and the rotating member is disposed coaxially with the transmitting gear. Here, it is necessary to dispose the rotating member at a side away from the driving motor with respect to the transmitting gear in order to arrange the rotating member at the end portion in the direction of the motor shaft. However, when the rotating member is protruded in the direction of the motor shaft with respect to the speed reduction mechanism by disposing the rotating member at the position just mentioned, the downsizing of the bending manipulating device relating to the dimension thereof in the direction of the motor shaft might be prevented.

As is apparent from the figures, particularly FIG. 2, the transmitting gear, which is the spur gear engaging with the movable gear, is arranged at a distance away from the driving motor in the direction of the motor shaft substantially identical to the distance from the driving motor to the movable gear in the direction of the motor shaft. Therefore, when the movable gear is arranged at a position away from the driving motor as in a conventional art, that is to say, when the movable gear is arranged at an end portion of the speed reduction mechanism in the direction of the motor shaft, the rotating member that is disposed at a side away from the driving motor with respect to the transmitting gear is to be disposed at a position that is protruded from the speed reduction mechanism with respect to the motor shaft direction. Consequently, the size of the bending manipulating device is reduced.

In order to alleviate the above inconveniences, the movable gear is arranged between the driving motor and the fixed gear in the present embodiment, unlike the general speed reduction mechanism that uses the planetary gears. Then, a space only for the fixed gear is held between a position where the transmitting gear is to be arranged and a position of the end portion of the speed reduction mechanism relating to the direction of the motor shaft, and the movable member is disposed at the space. By employing the configuration described above, the dimension of the bending manipulating device including the movable member in the direction of the motor shaft can be set substantially the same as the dimension of the speed reduction mechanism. Hence, there is an advantage that the bending manipulating device can be downsized with respect to the direction of the motor shaft, compared to a general speed reduction mechanism that uses the planetary gears.

Further, a center of the rotation of the speed reduction mechanism and a center of the rotation of the motor are set to equal to each other, and each of the motors is attached outside of the upward and downward speed reduction mechanism and the leftward and rightward speed reduction mechanism. Consequently, wiring inside the manipulating portion is facilitated as well as exchange of the motor can be performed easily by detaching the exterior case body.

Further, by manipulating the manipulating lever provided at the manipulating portion, the fixed gear that is rotatably disposed can be switched to the fixed gear that is disposed literally not to rotate or to the movable gear that rotates. Consequently, when the motor runaway is caused, bending of the bendable portion with an amount more than sufficient can be prevented by switching the disposing state of the fixed gear to the rotating state by manipulating the manipulating lever.

Further, the endoscope is configured so that the manipulation of the manipulating lever achieves the disengagement of the engaging portion from the engaging groove and the removal of the pressing force applied onto the friction member, without the need of special element as a mechanism for switching between the state of the fixed gear and the fixed state to the rotatable state. Consequently, the manipulating portion which is employed to deal with the motor runaway can be downsized and made at low cost.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an insertion portion that has a bendable portion including plural bending pieces connected with each other; and
a bending manipulating device that bends the insertion portion by rotating a traction member extending from the bendable portion and includes
a rotating member rotating with the traction member engaged,
a driving motor that rotates the rotating member, and
a speed reduction mechanism that transmits the driving force of the driving motor to the rotating member, the speed reduction mechanism including a sun gear rotated by the driving force of the motor and having first external teeth, plural planetary gears each having second external teeth engaging with the first external teeth, a first gear having first internal teeth engaging with the second external teeth, and a second movable gear having second internal teeth engaging with the second external teeth, the number of second internal teeth being different from the number of first internal teeth.

2. The endoscope according to claim 1, wherein
a motor shaft of the driving motor is protruded in a direction orthogonal to a traction direction of the traction member, and the sun gear is provided on the motor shaft.

3. The endoscope according to claim 2, wherein
the second movable gear has third external teeth, and
the rotating member is provided with a transmitting gear having fourth external teeth engaging with the third external teeth.

4. The endoscope according to claim 3, wherein
the rotating member is formed coaxially with the transmitting gear and disposed away from the driving motor with respect to the transmitting gear, and
the second movable gear is disposed between the driving motor and the first gear.

5. The endoscope according to claim 1, wherein
an outside diameter of the second movable gear and an outside diameter of the driving motor are substantially the same.

6. The endoscope according to claim 1, wherein
an outside diameter of the second movable gear is smaller than an outside diameter of the driving motor.

7. The endoscope according to claim 1, wherein
the first gear is rotatably disposed, the endoscope further comprising
a switching mechanism that properly switches the first gear between a fixed state and a movable state.
